(19) United States
(12) Patent Application Publication
(10) Pub. No.: US 2020/0009169 A1
(43) Pub. Date: Jan. 9, 2020

(54) COMPOUNDS, COMPOSITIONS AND METHODS FOR PREVENTION OR TREATMENT OF LIVER, LIPID, AND GLUCOSE-RELATED CONDITIONS

(71) Applicant: Sivanaray Inc., San Diego, CA (US)

(72) Inventor: Nallagounder Kuppusamy, (US)

(73) Assignee: Sivanaray Inc., San Diego, CA (US)

(21) Appl. No.: 16/487,829

(22) PCT Filed: Feb. 20, 2018
(86) PCT No.: PCT/US2018/018806

§ 371 Date: Aug. 21, 2019

Related U.S. Application Data
(60) Provisional application No. 62/461,678, filed on Feb. 21, 2017.

(51) Int. Cl. A61K 31/7004; A61K 9/20; A61K 9/14; A61K 9/48

(52) U.S. Cl. CPC A61K 31/7004; A61K 9/48; A61K 9/14; A61K 9/20

(57) ABSTRACT

The present invention discloses a method to reduce elevated blood glucose levels in a subject, typically having (pre-)diabetes, steatohepatitis, obesity, and/or metabolic syndrome, when orally administered. Particularly preferred compositions include a set of active components consisting of two or more isolated and purified saccharides selected from galacturonic acid, glucuronic acid, galactose, arabinose, glucuronic acid, rhamnose, xylose, and mannose. The composition administered to a subject in need thereof an effective dose of the composition for a period of at least 6 weeks, wherein administration results in an at least 5% reduction of blood glucose.

This application claims priority to U.S. provisional patent application with the Ser. No. 62/461,678, which was filed Feb. 21, 2017.

### FIELD OF THE INVENTION

The field of the invention is compounds, compositions, and methods for prevention or treatment of liver and glucose-related conditions, and particularly metabolic syndrome and/or reduction of elevated/(pre-)diabetic blood glucose levels.

### BACKGROUND

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Currently the world faces an epidemic of several closely related conditions: obesity, metabolic syndrome, and type 2 diabetes (T2DM). The lipid profile of these patients and those with metabolic syndrome is characterized by the concurrent presence of qualitative as well as quantitative lipoprotein abnormalities: low levels of HDL, increased triglycerides, and prevalence of LDL particles that are smaller and denser than normal. Central to these conditions is the liver, one of the largest and most important organs in the body, playing a vital role in the removal of waste products from the blood, distribution and storage of essential nutrients, and breakdown of harmful substances such as alcohol and toxic chemicals. Given the many vital functions performed by the liver, conditions affecting the liver can have devastating effects, whether hereditary or caused by pathogens, exposure to toxins, and/or lifestyle conditions such as a high-fat diet, alcohol consumption, and excessive caloric intake.

For example, various disease and environmental factors result in nonalcoholic fatty liver disease (NAFLD) and nonalcoholic steatohepatitis (NASH) that are associated with over-accumulation of lipids, which can be marked by inflammation and scarring in the liver. While most people with NAFLD or NASH are asymptomatic, these and other liver diseases can ultimately result in cirrhosis of the liver, hepatic encephalopathy, acute kidney injury, or even death. Some lipid and liver-related diseases have also been linked to insulin resistance, diabetes, and cardiovascular disorders. It is also well documented that (pre-)diabetes is linked to fat accumulation in the liver, which makes it harder to control fasting glucose levels and makes the body more resistant to insulin.

A healthy liver, normal glucose levels and a normal lipid profile are crucial to the health and well being of complex organisms.

Therefore, there is a significant need for compounds, compositions, and methods for preventing and treating lipid, glucose, and liver related disorders, especially for those having minimum toxicity and adverse effects.

For example, several plant or fruit-derived polysaccharides and specific modified forms thereof have been marketed to treat NASH and other lipid or liver-related conditions. Galectin Therapeutics Inc. teaches use of modified polysaccharides synthesized from pectin for the treatment of NASH (e.g., U.S. Pat. No. 8,658,787). Still others have investigated whether polysaccharides derived from Zizyphys jujube cv. Shaanbeitanzao could alleviate high fructose-induced insulin resistance and dyslipidemia in mice (e.g., Zhao et al., Food Funct 2014 August;5(8):1771-8).

Notably, pectin and Jujube derived polysaccharides discussed in the '787 patent and Zhao include various types of sugars, and are soluble dietary fibers. While these polymeric compounds have been reported as beneficial in providing some liver protective qualities, they tend to have a laxative effect, causing stomach cramps and diarrhea, nausea or bloating in some cases. Furthermore, the amounts of these polymers required to produce a therapeutic effect can be inconvenient in terms of both volume and laxative effect.

In other examples, nutritional supplements and herbs to reduce (pre-)diabetic glucose levels are well known and include alpha-lipoic acid, chromium-containing formulations, omega-3-fatty acids, various polyphenols, coenzyme Q, selected vitamins (e.g., C, E, B₆), and zinc. While such supplements are at least suitable to assist in reaching a recommended daily uptake for these compounds, their benefits are often marginal (e.g., Med Princ Pract. 2015 May; 24(3): 201-215). Moreover, more serious side effects have been reported, including allergic reactions, competitive inhibition for absorption of other nutrients, and drug-nutrient interactions resulting in long-term adverse effect (e.g., Afr J Tradit Complement Altern Med 2014; 11(3): 248-258).

Thus, there is still a need for improved and effective compounds, compositions, and methods for preventing and treating lipid, glucose, and liver/pancreas related disorders, and especially for prevention and treatment of elevated blood glucose and dyslipidemia.

### SUMMARY OF THE INVENTION

The applicant surprisingly discovered that selected components present in naturally occuring polysaccharides are highly effective for treating various metabolic disorders that are associated with dysregulated blood lipid and glucose levels, and/or fatty liver (e.g., NASH), and especially for reducing elevated blood glucose levels. Compounds and compositions of the inventive subject matter preferably include combinatioons of certain isolated and purified monosaccharides with improved bioavailability and reduced adverse effects as compared to known polysaccharides containin such monosaccharides. Specific saccharide components have been found to be particularly effective when used in specified ratios.

In one aspect of the inventive subject matter, the inventors contemplate uses and methods of reducing elevated blood glucose in a subject that includes a step of formulating or providing a composition comprising a set of active components in a therapeutically effective amount, wherein the set of active components consists essentially of at least two isolated and purified monosaccharides selected from: galacturonic acid, glucuronic acid, galactose, arabinose, rhamnose, xylose, and mannose; and a further step of administering to a subject in need thereof an effective dose of the composition for a period of at least 6 weeks, wherein administration results in an at least 5% reduction of the elevated blood glucose.

In especially contemplated aspects, the set of active components consist essentially of isolated and purified galacturonic acid, isolated and purified galactose and optionally isolated and purified arabinose, or consist essentially of isolated and purified glucuronic acid, isolated and purified galactose, and optionally isolated and purified arabinose. In further contemplated compositions, the molar ratio of galactose to galacturonic acid is between 1 and 3:1, and optionally the molar ratio of arabinose to galacturonic acid is between 4 and 8:1. While not limiting to the inventive subject matter, the composition is formulated as a tablet or capsule that includes the set of active components. For example, the set of active components comprises at least 50 wt %, or at least 70 wt %, or at least 95 wt % of total active components in the composition, and/or each of the isolated and purified monosaccharides has a purity of at least 90% (GC). It is further generally preferred that the composition is non-toxic when administered in a rodent model at a dose of 1000 mg/kg/day for a period of four weeks.

In further contemplated aspects, the set of active components may consist essentially of at least four isolated and purified monosaccharides selected from: galacturonic acid, glucuronic acid, galactose, arabinose, rhamnose, xylose, and mannose, or may consist essentially of at least five isolated and purified monosaccharides selected from: galacturonic acid, glucuronic acid, galactose, arabinose, rhamnose, xylose, and mannose, or may consist essentially of isolated and purified galacturonic acid, isolated and purified glucuronic acid, isolated and purified galactose, isolated and purified arabinose, isolated and purified rhamnose, isolated and purified xylose, and isolated and purified mannose. Most typically, the effective dose is between 5-50 mg/kg per day, or between 10-25 mg/kg per day.

Additionally, it is contemplated that administration of the composition further results in a reduction of lipids in the liver of the individual, a reduction in blood triglycerides, a reduction in blood LDL level, a reduction in blood ALP level, and/or a reduction in glycosylated hemoglobin in blood. Typically, but not necessarily, the subject may have steatohepatitis, type 2 diabetes, and/or metabolic syndrome, and/or may have pre-diabetes or type 2 diabetes.

Viewed from a different perspective, the inventors therefore also contemplate the use of at least two isolated and purified monosaccharides selected from: galacturonic acid, glucuronic acid, galactose, arabinose, rhamnose, xylose, and mannose in the manufacture of a supplement or therapeutic drug for the reduction of elevated blood glucose. As noted above, contemplated monosaccharides especially include isolated and purified galacturonic or glucuronic acid, isolated and purified galactose, and optionally isolated and purified arabinose, and/or the molar ratio of galactose to galacturonic acid is between 1 and 3:1, and the molar ratio of arabinose to galacturonic acid is optionally between 4 and 8:1. It is further contemplated that the isolated and purified monosaccharides are formulated as a powder, capsule, or a tablet, and/or that the therapeutic drug comprises the isolated and purified monosaccharides in an amount of at least 50 wt %. Most typically, the supplement or therapeutic drug is administered in an amount of between 5-50 mg/kg per day.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 provides exemplary graphs comparing various initial biochemical parameters for animals treated with selected compositions and control animals.

FIG. 2 provides exemplary graphs comparing various biochemical parameters at the third week for animals treated with selected compositions and control animals.

FIG. 3 provides exemplary graphs comparing various biochemical parameters at the sixth week for animals treated with selected compositions and control animals.

FIG. 4 provides exemplary graphs comparing various biochemical parameters at the ninth week for animals treated with selected compositions and control animals.

FIG. 5 provides exemplary graphs comparing various biochemical parameters at the twelveth week for animals treated with selected compositions and control animals.

FIGS. 6A-6E provides exemplary graphs comparing various biochemical parameters at sacrifice after 2 week washout period for animals treated with selected compositions and control animals.

FIG. 7 shows photomicrographs of stained sections of livers from animals treated with selected compositions and control animals.

FIG. 8 shows photomicrographs of stained sections of kidneys from animals treated with selected compositions and control animals.

### DETAILED DESCRIPTION

The applicant has discovered that specific compositions can be prepared and used to treat a subject to lower elevated blood glucose levels, especially where such elevated levels are associated with or caused by obesity, metabolic syndrome, steatohepatitis, pre-diabetes, or type 2 diabetes. The term “elevated levels of blood glucose” or “elevated blood glucose” are used interchangeably herein and refer to blood glucose levels that are above the clinical reference ranges for normal. For example, the human ‘normal’ reference range for fasting glucose is below 110 mg/dl (typically between 72-108 mg/dl). Therefore, an elevated blood glucose is present in a human subject in the pre-diabetic range (between 110-125 mg/dl), and the diabetic range (above 125 mg/dl). Remarkable, and as is shown in more detail further below, contemplated compositions and formulations were able to reduce blood glucose at least 10 mg/dl, or at least 20 mg/dl, or at least 30 mg/dl, or at least 40 mg/dl, or at least 50 mg/dl, or at least 60 mg/dl, or at least 70 mg/dl, or at least 80 mg/dl, or at least 90 mg/dl, or at least 100 mg/dl, or at least 120 mg/dl, or at least 150 mg/dl. Consequently, it should be noted that contemplated compositions and formulations are able to reduce blood glucose by at least 10%, or at least 20%, or at least 30%, or at least 35%, or at least 40%, or at least 45%, or at least 50% as compared to the elevated glucose value (e.g., of an individual having metabolic syndrome, steatohepatitis, pre-diabetes, type 2 diabetes, or obesity).

Most notably, contemplated compositions have also shown to be effective in lowering HOMA-IR values, blood cholesterol, triglyceride, and free fatty acid levels, fat accumulation in liver, and further lowerwed various liver/metabolic disease associated markers, including liver oxidative stress markers, plasma ALT levels, plasma ALP levels, plasma hs-CRP levels, plasma creatinine levels, plasma AST levels, plasma PTX3 levels, plasma leptin levels, plasma MCP-1 levels, and/or plasma insulin levels.

For example, and as described in more detail below, contemplated compositions are typically formulated as orally administered compositons (e.g., in dry form as a tablet, capsule, or in liquid form dissolved in an aqueous carrier) that comprises a set of active components in a therapeutically effective amount. It is especially preferred that the set of active components will consist essentially of at least two isolated and purified monosaccharides that are selected from the group of galacturonic acid, glucuronic acid, galactose, arabinose, rhamnose, xylose, and mannose (and in some cases even glucose). For example, preferred combinations of monosaccharides include glucuronic acid and galactose, or galactose and galacturonic acid, which may further include arabinose. In other preferred examples, formulations may include galacturonic acid, galactose, and arabinose (most preferably in a molar ratio of 1:2:3), or galacturonic acid, galactose, arabinose, and glucuronic acid (most preferably in a molar ratio of 1:2:3:0.5), or galactose, arabinose, and glucuronic acid (most preferably in a molar ratio of 2:3:1). Most typically, administration will be daily at the effective dose, for example 5-50 mg/kg per day, for a period of at least seven days, two weeks, four weeks, six weeks, 9 weeks, twelve weeks, or even longer.

Thus, it should be appreciated that effective compositions do not need to include all of galacturonic acid, glucuronic acid, galactose, arabinose, rhamnose, xylose, mannose, and glucose, but may include any subsets of these monosaccharides. Most preferably, however, contemplated combinations will include two monosaccharides, and especially an uronic acid and a non-uronic acid (e.g., galacturonic acid or glucuronic acid and galactose). Additionally, it should be appreciated that effective compositions could further or alternatively include other monosaccharides, and especially one or more of rhamnose, arabinose, glucose, xylose, and mannose. Indeed remarkable efficacy has been shown with various combinations of these monosaccharides in various molar ratios, and the selection of saccharides may be driven by the result desired and the condition to be treated, for example, as guided by the examples disclosed herein.

The isolated and purified saccharides can have any suitable purity, but will preferably have a purity of between 40-100% (GC), for example between 70-100%, between 80-100%, between 90-100%, between 95-100%, and most preferably between 98-100%. Additionally, while the sugars studied were naturally occurring isolated and purified monomers, naturally occurring and synthetically produced monomers, dimers, oligomers (3-10 monosaccharide units), and polymers (more than 10 monosaccharide units) are also contemplated herein. For example, a suitable saccharide may be obtained from a natural source such as a plant, fruit or vegetable, and optionally fragmented by acid, alkaline or catalytic hydrolysis, enzymatic digestion, oxidative lysis or radiative lysis. Thus, monosaccharides may also be accompanied by oligosaccharides.

Where the saccharides are not monomers, heteromeric and homomeric saccharides are contemplated. It should be appreciated that the isolated and purified saccharides can comprise L-isomers, D-isomers, or a mixture of L-isomers and D-isomers. Most typically, preferred monosccahrises are D-isomers (and in some cases (e.g., arabinose) also L-isomers). In some embodiments, the saccharides can comprise alpha linkages, beta linkages, or a combination of alpha and beta linkages. In further contemplated embodiments, one or more of the (mono)saccharides may be esterified, methylated, acetylated, amidated, or otherwise modified, preferably with a nutritionally acceptable component. Additionally, the sugars can be present in open-chain or ring form (e.g., furanoses, pyranoses). Moreover, all neutraceutical and pharmaceutically acceptable salts and pro-drugs are expressly contemplated herein.

In some embodiments, two or more of the same or different saccharides, preferably monomers, can be combined and bound together by glycosidic linkages to make a backbone, oligomeric or polymeric structure. However, other linkages are also deemed suitable, particularly where the linkage is nutritionally or pharmaceutically acceptable (e.g., polylactides, PEG, glycols, diesters). For example, isolated and purified galacturonic acid, isolated and purified galactose, isolated and purified arabinose, and isolated and purified glucuronic monomers could be bound together by alpha or beta glycosidic linkages to form an hetero or homo oligomeric or polymeric structure wherein the molar ratio of galactose to galacturonic acid, and the molar ratio of galactose to galacturonic acid can be between 1:15 and 15:1, between 1:10 and 10:1, more preferably between 1:2 and 5:1, and even more preferably between 1:1 and 3:1, and the molar ratio of arabinose to galacturonic acid can be between 1:15 and 15:1, between 1:10 and 10:1, more preferably between 2:1 and 10:1, and even more preferably between 4:1 and 8:1, or between 5:1 and 7:1. Preferably, the oligomeric or polymeric structure will predominantly consist of (i.e., be at least 85% composed of) galacturonic acid, galactose, arabinose, glucuronic acid, xylose, glucose, mannose, rhamnose, or any subset or combinations thereof. The so formed oligomer or polymer can have between 3 and 10, between 10 and 100, or even between 100 and 1,000 (or even more) monomers connected in linear or branched forms.

The isolated and purified saccharides in contemplated compositions can be present in any suitable molar or weight ratio. In some preferred embodiments, each saccharide will be present in a molar ratio that is no greater than 20:1, more preferably no greater than 10:1, with respect to each of the other individual saccharides in the composition. As some non-limiting examples, the isolated and purified saccharides in contemplated compositions can e present in any of the molar ratios shown in Table 1.

TABLE 1

| Saccharide | Moles | Moles | Moles | Moles | Moles | Moles | Moles | Moles | Moles |
|---|---|---|---|---|---|---|---|---|---|
| Galacturonic | 1 | 1 | 1 | 1 | 0 | 1 | 1 | 1 | 1 |
| Acid | | | | | | | | | |
| Galactose | .2-1.5 | .2.-7 | 1-3 | 1-3 | .1-10 | .1-10 | .1-10 | .05-20 | .05-20 |
| Arabinose | .3-4.5 | .3-.8 | 1-5 | 4-8 | .1-10 | .1-10 | | .05-20 | |
| Glucuronic | 1 | 1 | 1 | 1 | 1 | 0 | 1 | 1 | 1 |
| Acid | | | | | | | | | |
| Rhamnose | .05-.5 | .05-.1 | .1-.6 | .5-1 | .1-10 | | | | |
| Glucose | .05-1 | | | 1-2 | .1-10 | | | | |
| Xylose | .5-4 | | | 4-6 | .1-10 | | | | |
| Mannose | .5-1.5 | | | .5-3.5 | .1-10 | | | | |

Before the inventor's discovery, it was unknown and unexpected that specific monomer components of naturally derived polysaccharides could also be highly effective for treating various metabolic and/or lipid or liver related disorders. Notably, while the existing literature reported examples that emphasized liver benefitting effects resulting from the use of polysaccharides per se, applicant was surprisingly able to identify effective monosaccharide components, and combine them in effective (and in certain cases synergistic) ratios to thereby obtain desirable biological effects. It should be appreciated that such finding is very unusual as other polymeric molecules (e.g., insulin polypeptide) exert specific biological effects only in the polymeric form and not in a combination of a subset of isolated and purified amino acids found in the polymer.

With respect to suitable amounts of contemplated set of actives in the composition, it should be recognized that the particular quantity will typically depend on the specific formulation, active ingredient, and desired purpose. Therefore, it should be recognized that the amount of actives in contemplated compositions will vary significantly. However, it is generally preferred that the set of actives is present in a minimum amount effective to deliver a therapeutic effect or to be achieved in vitro or in vivo.

However, it is generally preferred that the compositions according to the inventive subject matter are delivered in a therapeutically effective amount. The term “therapeutically effective amount” refers to the amount of the compound or composition that will elicit a biological or medical response of a tissue, system, animal or human that is being sought (e.g., reduction elevated blood glucose levels, reduction in body weight; reduction in a HOMA-IR value; reduction in a total cholesterol plasma level; reduction in fat accumulation in the liver; reduction in a liver oxidative stress marker level (e.g., MDA)). The optimum therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Typically, appropriate doses are normally from 0.01 to 500 mg/kg of body weight per day, more preferably from 0.1 to 100 mg, from 0.1 to 50 mg/kg/day, or from 3.5 to 50 mg/kg/day. While the formulations described herein were administered to animals at between 50-1,000 mg/kg body weight per day, the person skilled in the arts could calculate the human effective dosage from known Kₘ factors. Depending on the animal models used, the human effective dosage will typically be between 5-35%, more preferably 7-15% in humans of the animal effective dosage. Consequently, it is contemplated that the effective dose of the composition or set of active ingredients is between 0.5-50 mg/kg per day, or higher in some instaces. For example, effective doses will be between 0.5-5 mg/kg per day, or between 5-20 mg/kg per day, or between 10-25 mg/kg per day, or between 25-50 mg/kg per day, or between 50-100 mg/kg per day.

The administration of the suitable dose can be administered once per day, or can be spread out over the course of a day. For example, an effective dose of the composition can be divided and separately packaged as two to five capsules, tablets, powders or oral dissolve strips, and separately administered two to five times a day. Alternate day dosing or dosing once every several days may also be utilized. Depending on the particular use and structure, it is contemplated that the set of actives according to the inventive subject matter are present in the composition in an amount between 1 microgram to 10,000 milligram, more typically between 10-500 milligram, and most typically between 50 mg to 1000 mg per single dosage unit. The compositions according to the inventive subject matter may be administered using various routes, but is preferably administered orally in any orally acceptable dosage form including, but not limited to, capsules, powders, tablets, troches, elixirs, suspensions, syrups, wafers, chewing gums, aqueous suspensions or solutions.

The pharmaceutical, neutraceutical and supplement preparations can be made following the conventional techniques of pharmacy involving milling, mixing, granulation, and compressing, when necessary, for tablet forms; or milling, mixing and filling for hard gelatin capsule forms. When the dosage unit form is a capsule, it may additionally contain a pharmaceutically acceptable carrier, such as a liquid carrier (e.g., a fatty oil). Other dosage unit forms may contain other various materials which modify the physical form of the dosage unit, such as, for example, a coating. Thus, tablets or pills may be coated with sugar, shellac, or other enteric coating agents. A liquid or syrup may contain, in addition to the active ingredients, sucrose as a sweetening agent and certain preservatives, dyes and colorings and flavors. Materials used in preparing these various compositions should be pharmaceutically or veterinarally pure and non-toxic in the amounts used. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation.

Although oral compositions may be preferred, all commercially suitable routes of administration are contemplated, including oral, parenteral, inhalation, topical, rectal, nasal, or via an implanted reservoir, wherein the term “parenteral” as used herein includes subcutaneous, intravenous, intramuscular, intraarticular, intrasynovial, intrathecal, intrahepatic, intralesional, and intracranial administration (typically injection or infusion).

For therapeutic or prophylactic purposes, contemplated compounds are ordinarily combined with one or more excipients appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, or polyvinyl alcohol, and then tableted or encapsulated for convenient administration. Such capsules or tablets may contain a controlled-release formulation as may be provided in a dispersion of active compound in hydroxypropylmethyl cellulose.

While compositions are preferably in pharmaceutical form, nutraceuticals such as dietary supplements, nutritional supplements and medical foods are also contemplated. The nutraceuticals can optionally be used in combination with foods, beverages, spices, condiments, salad dressings and any other goods where soluble starches or fibers are used.

Depending on the particular purpose, it should also be recognized that contemplated compounds, compositions or sets of active compounds may be combined (in vivo, or in a therapeutic formulation or administration regimen) with at least one other therapeutically active agent to additively or synergistically provide a therapeutic or prophylactic effect. Concentrations of the other therapeutically active ingredients are typically at or preferably below those recommended for stand-alone administration, however, higher concentrations are also deemed suitable for use herein. Additional ingredients are considered supplemental or additive, and may or may not be present in synergistic quantities with respect to weight loss, treating a condition associated with, or lowering, a HOMA-IR value, a total cholesterol plasma level, fat accumulation in liver, a liver oxidative stress marker level, a plasma glucose level, a plasma urea level, a plasma free fatty acid level, a plasma triglyceride level, a plasma ALT level, a plasma ALP level, a plasma hs-CRP level, a plasma creatinine level, a plasma AST level, a plasma PTX3 level, a plasma leptin level, a plasma MCP-1 level, a plasma insulin level, and a plasma LDL level. Therefore, additional ingredients could include, for example, drugs approved for use in reduction of cholesterol (e.g., various statins or fibrates) and/or blood glucose (e.g., sulfonylureas, biguanides, thiszolidinediones, DPP-4 inhibitors, etc.), anti-inflammatory drugs, and especially non-NSAIDs, and/or nutritional supplements used for treatment of liver diseases (e.g., sylimarin extracts), dyslipidemia (e.g., betaine, red rice yeast, etc.) and/or high blood sugar (e.g., alpha-lipoic acid, chromium-picolinate, omega-3-fatty acids, green tea polyphenols, coenzyme Q, vitamins, etc.).

The effectiveness of applicant's compositions and methods have been supported by several experimental studies performed on animals, including control groups and animals with induced type 2 diabetes (T2DM), as provided in more detail below. In this series of studies, various combinations of isolated and purified monosaccharides were administered orally to these animals. In addition, further suitable compositions and compounds are disclosed in applicant's International patent application WO2017/023703 and International patent application with the serial number PCT/US18/13632.

### Experiments

Male Sprague-Dawley (SD) rats weighing 110-120 g were procured (IAEC 17/09) from the Central Animal Facility of National Institute of Pharmaceutical Education and Research, S.A.S. Nagar, India and kept for acclimatization in the institute's test facility, National Toxicology Centre till they attained weight of 150-160 g. Further, they were divided in following groups and treated with different formulations of (SN-09, SN-10, SN-11, SN-12, SN-13 and SN-14) for 12 weeks followed by changing the diet to NPD and discontinuation of above formulations for 2 weeks before sacrifice. Table 2 lists the ingredients for each of the different formulations used in this study.

TABLE 2

| | SN9 | SN10 | SN11 | SN12 | SN13 | SN14 |
|---|---|---|---|---|---|---|
| Saccharide | Moles | Moles | Moles | Moles | Moles | Moles |
| Galacturonic | — | — | — | 1 | 1 | 1 |
| Acid | | | | | | |
| Galactose | 2 | 2 | 2 | 2 | 2 | 2 |
| Arabinose | 3 | — | — | 3 | — | — |
| Glucuronic | 4 | 1 | 1 | — | — | — |
| Acid | | | | | | |
| Rhamnose | — | — | — | — | — | — |
| Glucose | — | — | — | — | — | — |
| Xylose | — | — | — | — | — | — |
| Mannose | — | — | — | — | — | — |

Animals were grouped into groups of 8 and each group received the formulations at the dosage indicated in Table 3, with the treated animals in SN-09 to SN-14 being T2DM animals, and T2DM animals not receiving any formulation of SN-09 to SN-14.

TABLE 3

| Group | Dose | N |
|---|---|---|
| Control | NA | 8 |
| T2DM | HFD + STZ (35 mg/kg, intraperitoneal) | 8 |
| SN-09 | 400 mg/kg, per oral | 8 |
| SN-10 | 400 mg/kg, per oral | 8 |
| SN-11 | 200 mg/kg, per oral | 8 |
| SN-12 | 400 mg/kg, per oral | 8 |
| SN-13 | 400 mg/kg, per oral | 8 |
| SN-14 | 200 mg/kg, per oral | 8 |

Clinical tests for analytes were performed after overnight fasting, and blood was collected with EDTA and centrifuged at 7000 rpm for 7 min, and plasma was collected and used for biochemical assays and ELISAs following the manufacturers instructions. For the analytes Glucose, LDL-C, HDL-C, Triglycerides, Cholesterol, SGOT (AST), SGPT (ALT), ALP, and Creatinine, the tests were performed using Laboratory Ananlyzers from Accurex Biomedical Pvt. Ltd., Mumbai, India. Insulin was determined using a test kit from Crystal Chem Inc., IL, USA, and TGF-β1 was determined using a test kit from Abcam, UK.

Diabetic mice were generated following known protocols. More specifically, male, SD, rats weighing 150-160 g were fed with high fat diet [(HFD), prepared in-house, in form of round ball, composition as per Table 4) for 4 weeks followed by streptozotocin (STZ) injection (35 mg/kg, intraperitoneal) at the end of the 4^{th} week to induce the type 2 diabetes mellitus (T2DM).

After 2 weeks, blood glucose was estimated, and only animals with blood glucose 250 mg/dL were considered diabetic and subsequently randomized into different groups: Control, T2DM, and T2DM treated with different formulations of (SN-09, SN-10, SN-11, SN-12, SN-13 and SN-14). The plasma biochemical parameters were measured at 4^{th} week (before STZ injection), 2 weeks after STZ injection during model development; 3^{rd}, 6^{th}, 9^{th} and 12^{th} week of treatment and at the end of study after shifting animals to NPD for 2 weeks after discontinuing the formulations.

TABLE 4

| | Amount |
|---|---|
| Ingredients | (g/kg) |
| Powdered NPD | 365 |
| Lard | 310 |
| Casein | 250 |
| Cholesterol | 10 |
| Vitamin and mineral mix | 60 |
| dl-Methionine | 03 |
| Yeast powder | 01 |
| Sodium chloride | 01 |

Histopathology: The kidney and liver were fixed in 10% neutral buffer formalin, dehydrated gradually in ethanol and xylene, then embedded in paraffin and 5 μm thin sections were used for histopathological analysis. The rehydrated sections were stained using haematoxylin and eosin (H&E), mounted with DPX and examined under the microscope (Olympus BX51 microscope, Tokyo, Japan). Histological alteration such as diameter of pancreatic islets and capsular space in kidney tissue were evaluated and quantified using Image J software. For liver, sections were scored for extent of steatosis where “++++” meaning highest and “-” meaning no steatosis.

Statistical Analysis: All data were expressed as mean ±SEM. For statistical significance, means of two groups were compared using Student's t-test and more than two groups using one-way ANOVA followed by Tukey's post hoc test using GraphPad Prism v5.01 (GraphPad Software. Inc., CA, USA). Data from the T2DM group was compared with the control group and all treated groups were compared to T2DM group. In the raw data, outliers were not included for plotting the graphs. Outliers were identified using Tukey's interquartile range method using IBM SPSS Statistics v20 (IBM Corporation, NY, USA).

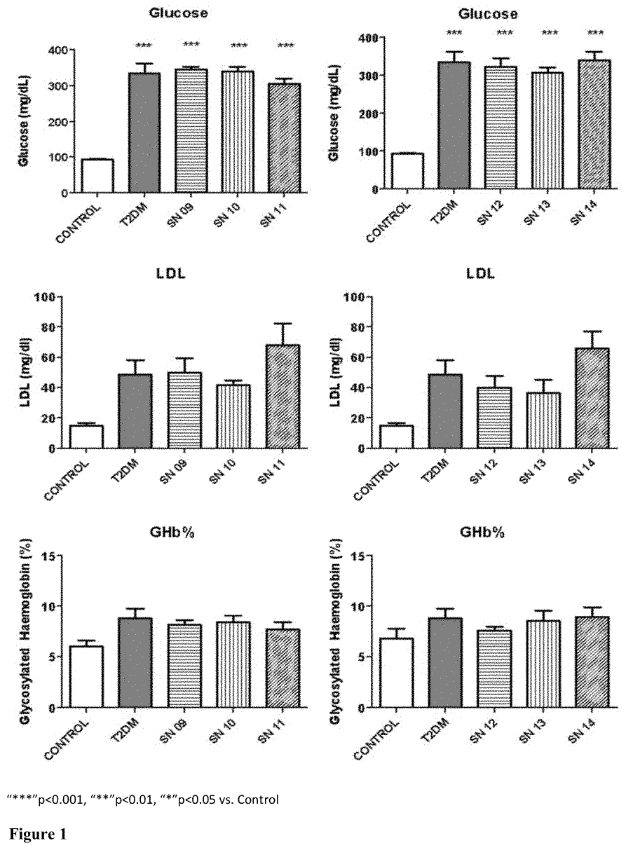

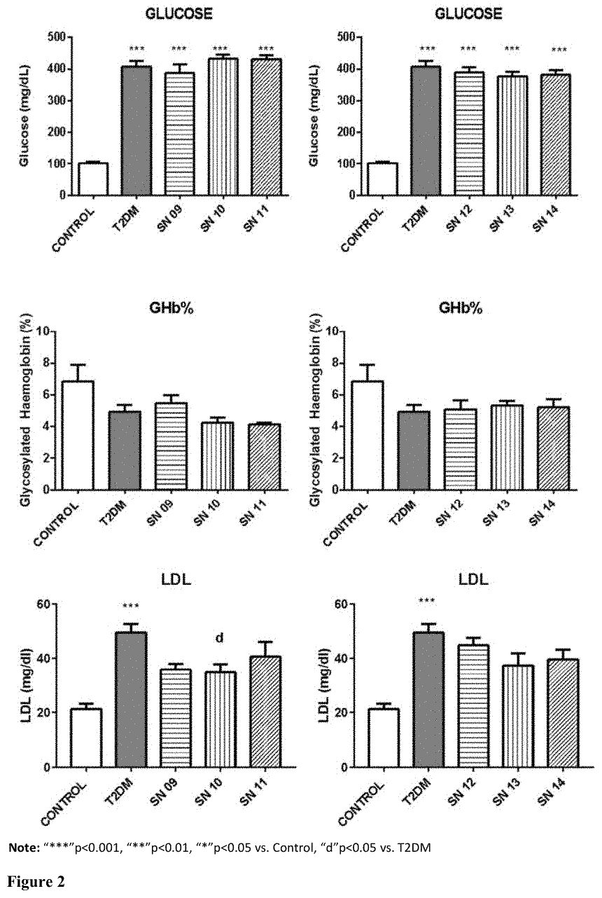

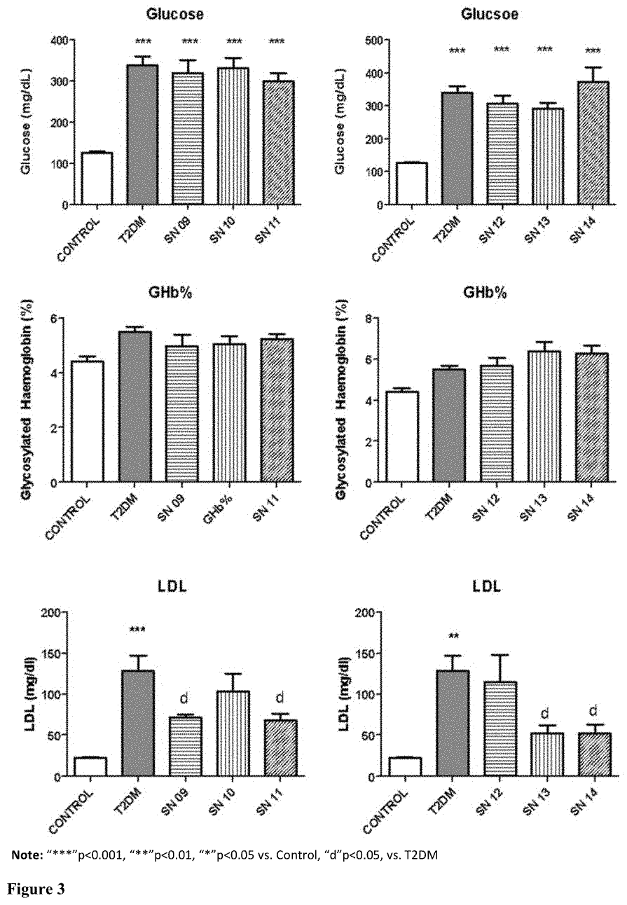

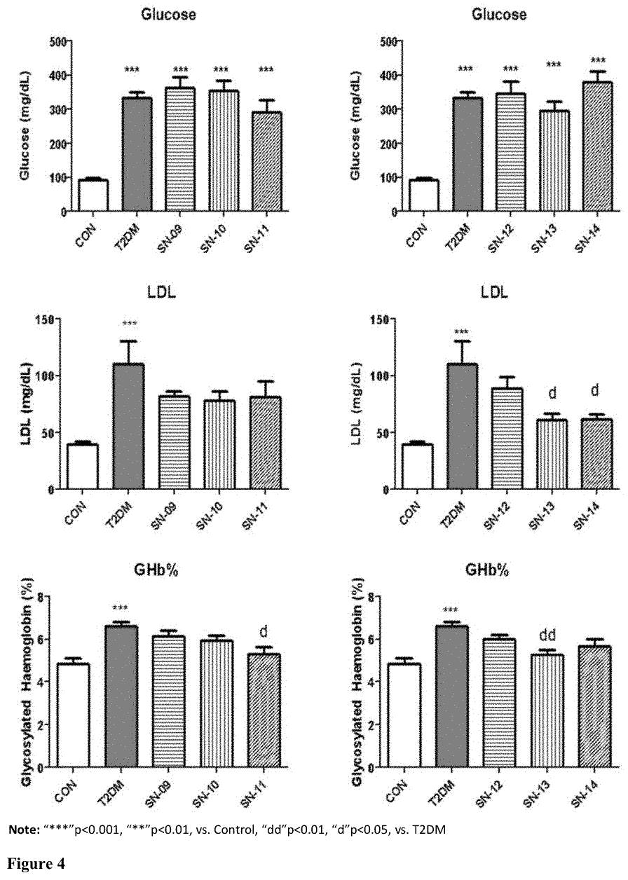

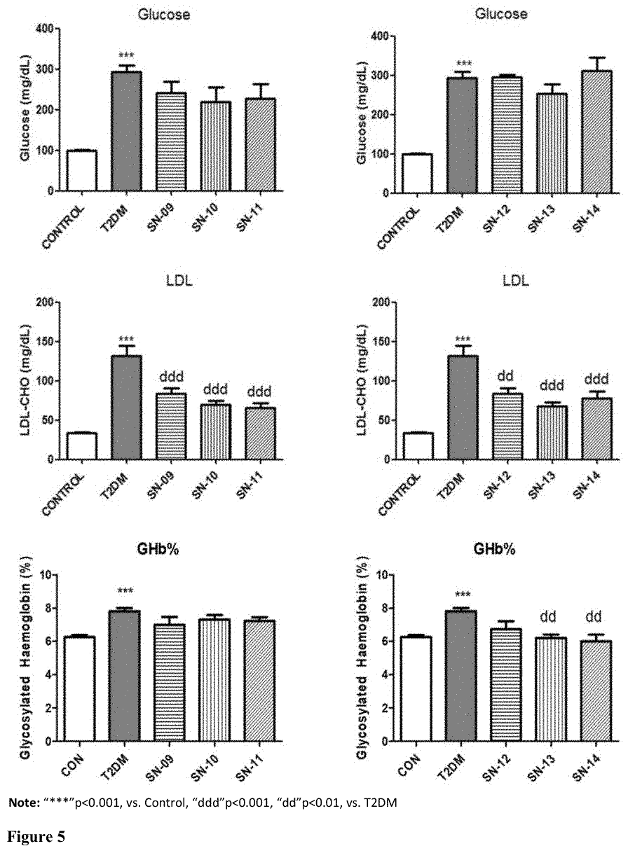

As can be seen from the data in FIG. 1, T2DM animals and all animals treated with the formulations SN-09 to SN-14 had significantly elevated glucose levels relative to normal control at week 0. Similarly, LDL-cholesterol levels were significantly elevated in the T2DM animals and all animals treated with the formulations SN-09 to SN-14. Glycosylated hemoglobin was within the expected variation and did not show significantly differences between control, T2DM, and treated animals. At the third week as shown in FIG. 2, results were substantially similar to those at week 0, with the exception of a statistically significant reduction of LDL cholesterol in SN-10 treated animals and a trend towards lowering LDL in all treated animals. This trend became more evident at the sixth week, where LD cholesterol was significantly reduced in most of the treated animals as compared to T2DM control as is shown in FIG. 3. Substantially similar results are depicted for the ninth week as is shown in the graphs of FIG. 4. Here, it was also noted that the degree of glycosylated hemoglobin was reduced to a statistically significant degree in at least some of the treated animal groups, which suggested that contemplated compositions also achieved significant reduction in blood glucose. However, at that time the glucose measurements were not in agreement with that hypothesis. Notably, LDL cholesterol and glycosylated hemoglobin data showed continued and statistically significant improvements at the twelfth week as is shown in the graphs of FIG. 5. This disparity in apparent high glucose levels and low glycosylated hemoglobin led the applicant to investigate whether or not the orally administered compounds could in fact interfere with the measurements. To that end, test reactions were run with plasma of a control animal plus one of the formulations administered (SN-13, SIVISBRM5), and the results are shown in Table 5.

TABLE 5

| | | Glucose level |
|---|---|---|
| | Method | (mg/dL) |
| Net | Glucose reagent 1 mL + CON Plasma 10 μL | 110 |
| volume | Glucose reagent 1 mL + 50 μL Water + CON Plasma 10 μL | 106 |
| 1060 μL | Glucose reagent 1 mL + 40 μL Water + 10 μL SIVISBRM 5 + | 163 |
| | CON Plasma 10 μL | |
| | Glucose reagent 1 mL + 30 μL Water + 20 μL SIVISBRM 5 + | 208 |
| | CON Plasma 10 μL | |
| | Glucose reagent 1 mL + 20 μL Water + 30 μL SIVISBRM 5 + | 247 |
| | CON Plasma 10 μL | |
| | Glucose reagent 1 mL + 10 μL Water + 40 μL SIVISBRM 5 + | 304 |
| | CON Plasma 10 μL | |
| | Glucose reagent 1 mL ++ 50 μL SIVISBRM 5 + CON | 348 |
| | Plasma 10 μL | |
| | Glucose reagent 1 mL + T2DM Plasma 10 μL | 328 |
| | Glucose reagent 1 mL + 50 μL Water + T2DM Plasma 10 | 320 |
| | μL | |
| | Glucose reagent 1 mL + 40 μL Water + 10 μL SIVISBRM 5 + | 385 |
| | T2DM Plasma 10 μL | |
| | Glucose reagent 1 mL + 30 μL Water + 20 μL SIVISBRM 5 + | 439 |
| | T2DM Plasma 10 μL | |
| | Glucose reagent 1 mL + 20 μL Water + 30 μL SIVISBRM 5 + | 479 |
| | T2DM Plasma 10 μL | |
| | Glucose reagent 1 mL + 10 μL Water + 40 μL SIVISBRM 5 + | 519 |
| | T2DM Plasma 10 μL | |
| | Glucose reagent 1 mL + 50 μL SIVISBRM 5 + T2DM | 580 |
| | Plasma 10 μL | |
| Without | Glucose reagent 1 mL + 50 μL Water + 10 μL (2.34 mg) | 38 |
| plasma | SIVISBRM 5 | |
| | Glucose reagent 1 mL + 40 μL Water + 20 μL | 112 |
| | (4.86 mg)SIVISBRM 5 | |
| | Glucose reagent 1 mL + 30 μL Water + 30 μL (7.02 mg) | 150 |
| | SIVISBRM | |
| | Glucose reagent 1 mL + 20 μL Water + 40 μL (9.36 mg) | 190 |
| | 2SIVISBRM 5 | |
| | Glucose reagent 1 mL + 10 μL Water + 50 μL | 249 |
| | (11.7 mg)SIVISBRM 5 | |

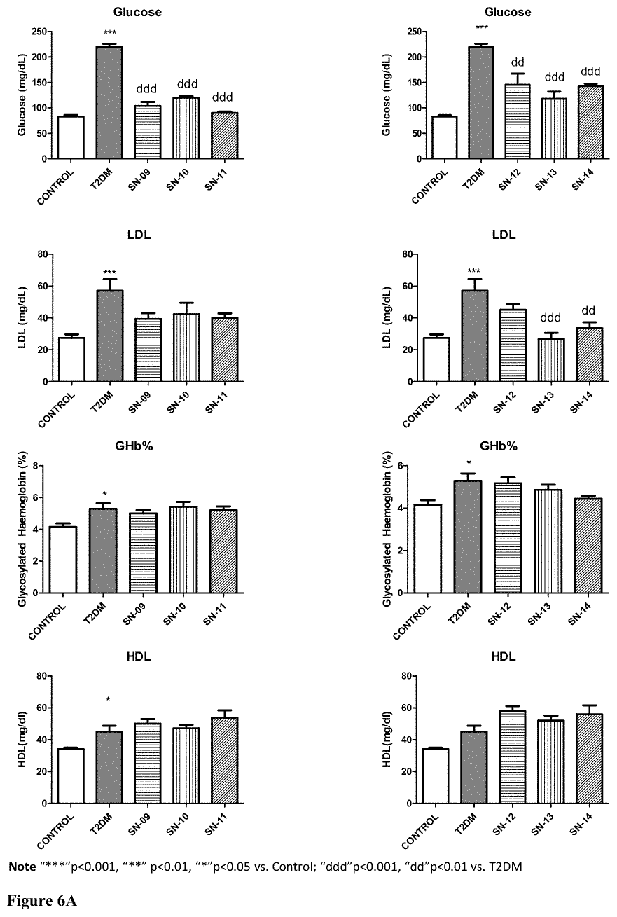

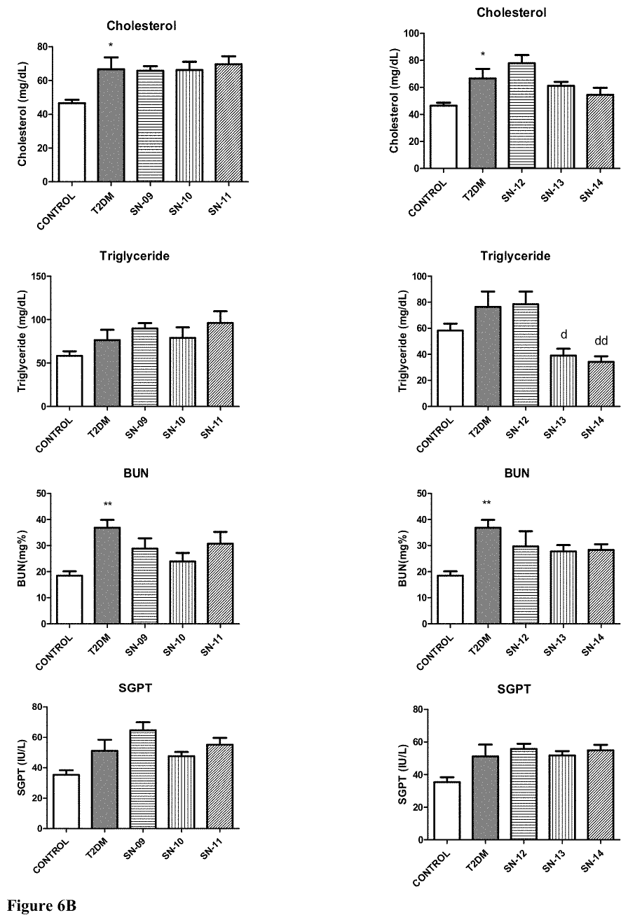

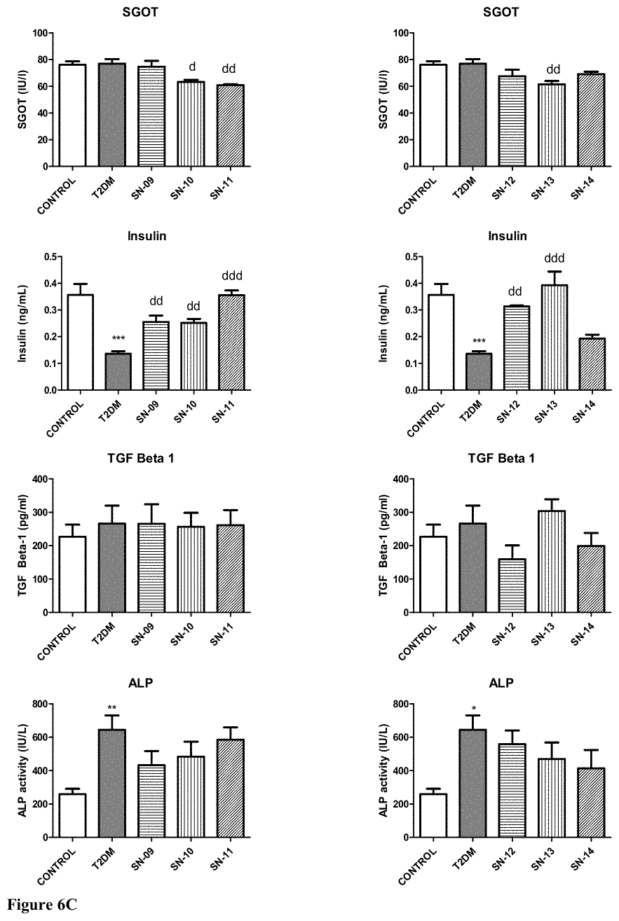

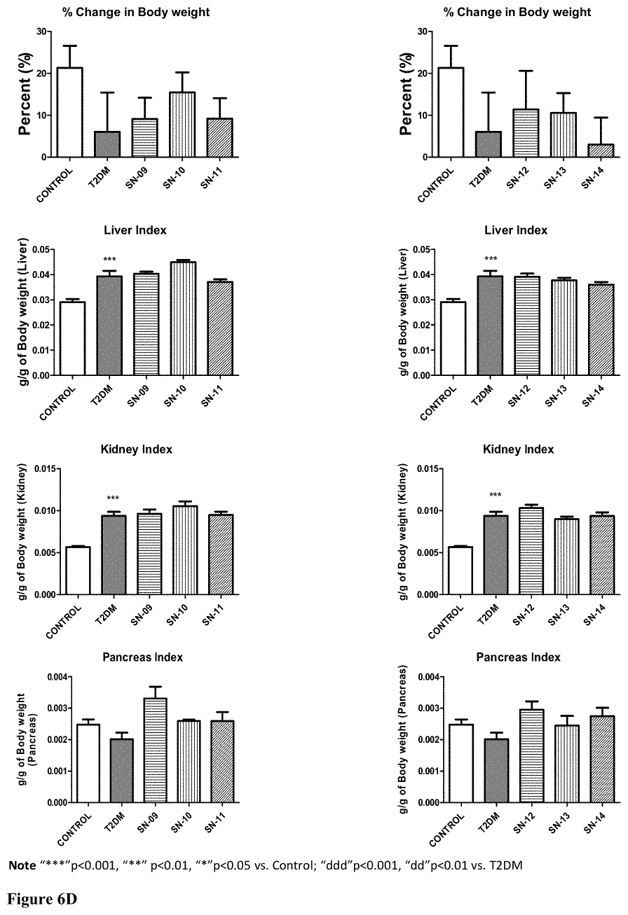

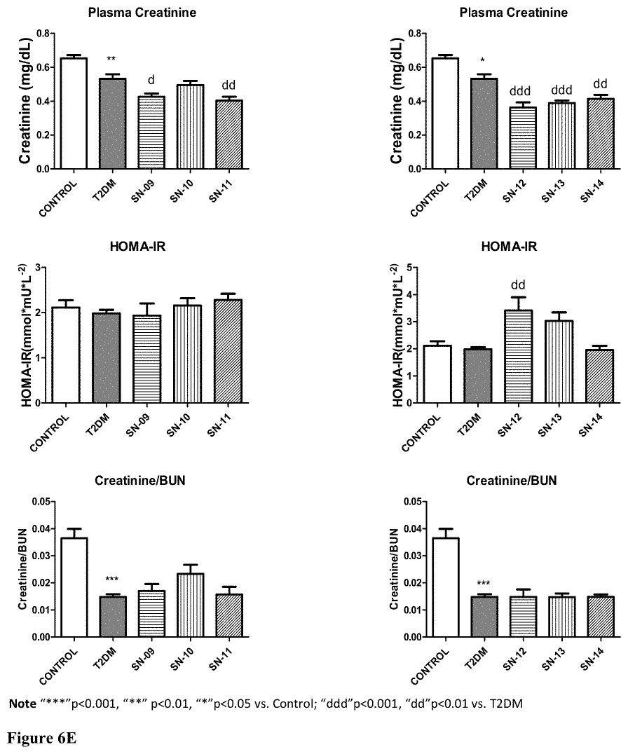

Notably, the treatment formulation skewed accurate results for both control animal plasma and T2DM animal plasma in a dose-dependent fashion. Based on this unexpected observation, the applicant continued maintenance of all animals using normal pellet diet, but discontinued treatment with the formulations as described above. After a two week washout period, numerous biochemical parameters were determined and the results are depicted in the graphs of FIG. 6A to FIG. 6E. Most surprisingly, after the two week washout period of the treatment formulation, strong and statistically significant differences were observed for blood glucose for all animals treated and tested. Indeed, contemplated compositions and formulations reduced blood glucose at least 10 mg/dl, or at least 20 mg/dl, or at least 30 mg/dl, or at least 40 mg/dl, or at least 50 mg/dl, or at least 60 mg/dl, or at least 70 mg/dl, or at least 80 mg/dl, or at least 90 mg/dl, or at least 100 mg/dl, or at least 120 mg/dl, or at least 150 mg/dl. Viewed from another perspective, it should be appreciated that the contemplated compositions and formulations reduced blood glucose at least 10%, or at least 20%, or at least 30%, or at least 35%, or at least 40%, or at least 45%, or at least 50% from the elevated glucose value. Thus, contemplated formulations not only significantly improved various aspects of dyslipidemia, but also provided a durable effect of at least two weeks in reducing elevated blood glucose after treatment was discontinued (see FIG. 6A).

Moreover, as is shown in FIG. 6B, triglycerides were also significantly reduced in some of the treated animals, as well as some of the liver damage markers (SGOT) as can be taken from FIG. 6C. Remarkably, contemplated compositions also appeared to at least partially restore pancreatic function (insulin) as is also evident from the graphs of FIG. 6C. Furthermore, kidney function significantly improved as measured by plasma creatinine in almost all of the treated animals (FIG. 6E).

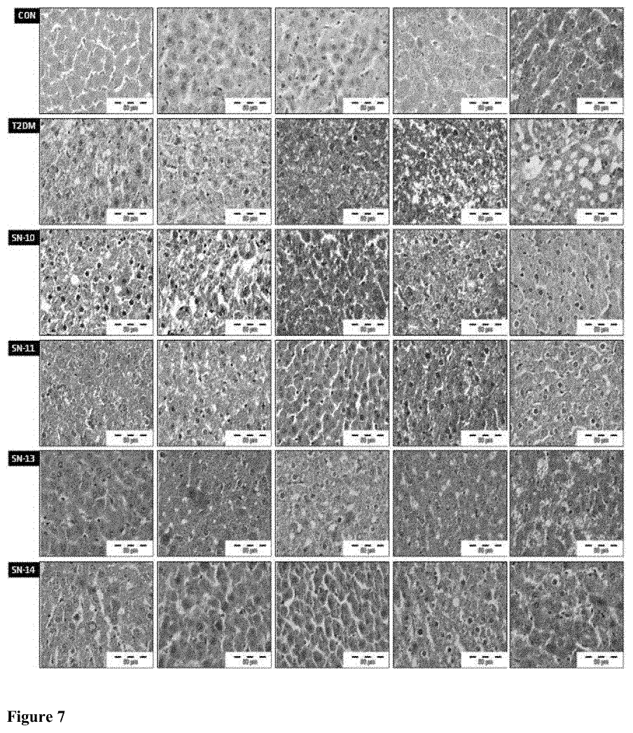

In addition, notable improvements were also observed in histopathology where the animals were treated with contemplated compositions. More specifically, FIG. 7 shows photomicrographs of liver sections of control animals (CON) with no evidence of steatosis. In contrast, T2DM animals had significant fatty globular deposits (T2DM), while the treated animals had a profound improvement over T2DM (SN10/11/13/14). Table 6 is a qualitative assessment of the degree of steatosis (where “++++” refers to highest degree and “-”refers to no steatosis).

TABLE 6

| Group | Animal | Scoring (for steatosis) |
|---|---|---|
| Control | 1 | − |
| | 2 | − |
| | 3 | − |
| | 4 | − |
| | 5 | − |
| T2DM | 1 | ++++ |
| | 2 | ++++ |
| | 3 | +++ |
| | 4 | +++ |
| | 5 | ++++ |
| SN-10 | 1 | ++ |
| | 2 | +++ |
| | 3 | + |
| | 4 | + |
| | 5 | + |
| SN-11 | 1 | ++ |
| | 2 | ++ |
| | 3 | + |
| | 4 | + |
| | 5 | + |
| SN-13 | 1 | + |
| | 2 | + |
| | 3 | ++ |
| | 4 | + |
| | 5 | ++ |
| SN-14 | 1 | ++ |
| | 2 | ++ |
| | 3 | + |
| | 4 | ++ |
| | 5 | + |

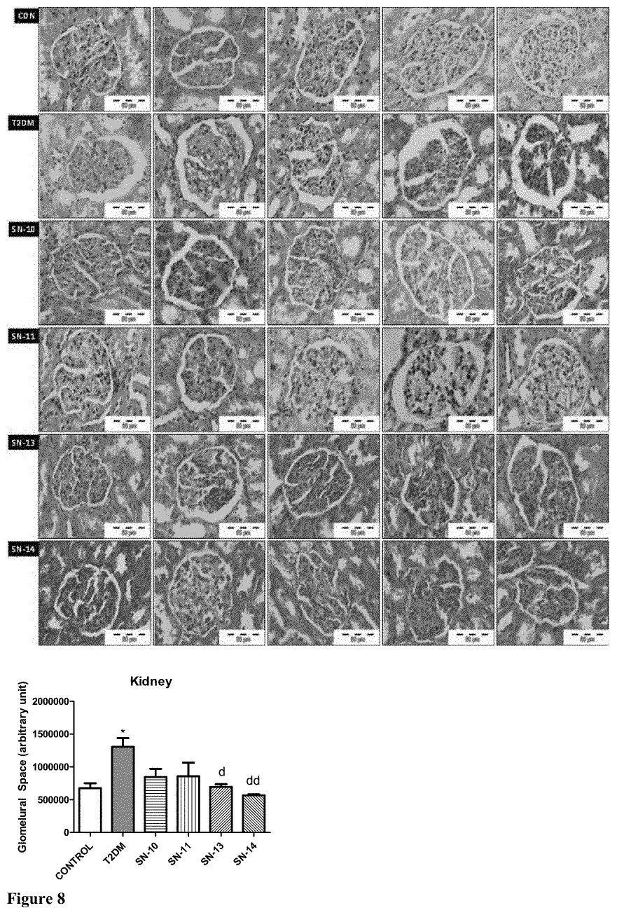

Similar improvements were also observed in histopathological analyses of the kidney as is shown in FIG. 8. Here, statistically significant improvements were demonstrated for several formulations and a strong trend for the remaining formulations with respect to the glomerular space. These results are corroborated by at least some of the biochemical data, and especially the data for creatinine clearance.

Additionally, inventors also observed that administration of 1000 mg/kg/day of a formulation including some or all of the monosaccharides described herein to animals fed a NPD diet and not injected with STZ displayed no significant deviations in basal parameters, activity level, feed intake, weight gain, water intake, coagulation time, glucose, triglycerides, cholesterol, high density lipoprotein (HDL), low density lipoprotein (LDL), liver function, oxidative stress markers, morphometry of liver, kidney and histopathology of liver relative to a group of animals fed a NPD diet, not injected with STZ, and not receiving any treatment formulation. This lack of significant difference indicated the formulation can safely be consumed at high doses without any major side effects. Similar results are expected with respect to formulations including glucuronic acid.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Moreover, and unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

The discussion herein provides example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

It should be apparent, however, to those skilled in the art that many more modifications besides those already described are possible without departing from the inventive concepts herein. The inventive subject matter, therefore, is not to be restricted except in the spirit of the disclosure. One skilled in the art will recognize many methods and materials similar or equivalent to those described herein, which could be used in the practice of the present invention. Indeed, the present invention is in no way limited to the methods and materials described.

Moreover, in interpreting the disclosure all terms should be interpreted in the broadest possible manner consistent with the context. In particular the terms “comprises” and “comprising” should be interpreted as referring to the elements, components, or steps in a non-exclusive manner, indicating that the referenced elements, components, or steps can be present, or utilized, or combined with other elements, components, or steps that are not expressly referenced.

What is claimed is:

1. A method of reducing elevated blood glucose in a subject, comprising:
formulating or providing a composition comprising a set of active components in a therapeutically effective amount;
wherein the set of active components consists essentially of at least two isolated and purified monosaccharides selected from: galacturonic acid, glucuronic acid, galactose, arabinose, rhamnose, xylose, and mannose; and
administering to a subject in need thereof an effective dose of the composition for a period of at least 6 weeks, wherein administration results in an at least 5% reduction of blood glucose.

2. The method of claim 1, wherein the set of active components consist essentially of isolated and purified galacturonic acid, isolated and purified galactose and optionally isolated and purified arabinose.

3. The method of claim 1, wherein the molar ratio of galactose to galacturonic acid is between 1 and 3:1, and optionally wherein the molar ratio of arabinose to galacturonic acid is between 4 and 8:1.

4. The method of claim 1, wherein the composition is formulated as a tablet or capsule that includes the set of active components.

5. The method of claim 1, wherein the set of active components comprises at least 70 wt % of the composition.

6. The method of claim 1, wherein the set of active components comprises at least 95 wt % of total active components in the composition.

7. The method of claim 1, wherein each of the isolated and purified monosaccharides has a purity of at least 90% (GC).

8. The method of claim 1, wherein the composition is non-toxic when administered in a rodent model at a dose of 1000 mg/kg/day for a period of four weeks.

9. The method of claim 1, wherein the set of active components consist essentially of at least four isolated and purified monosaccharides selected from: galacturonic acid, glucuronic acid, galactose, arabinose, rhamnose, xylose, and mannose.

10. The method of claim 1, wherein the set of active components consist essentially of at least five isolated and purified monosaccharides selected from: galacturonic acid, glucuronic acid, galactose, arabinose, rhamnose, xylose, and mannose.

11. The method of claim 1, wherein the set of active components consist essentially of isolated and purified galacturonic acid, isolated and purified glucuronic acid, isolated and purified galactose, isolated and purified arabinose, isolated and purified rhamnose, isolated and purified xylose, and isolated and purified mannose.

12. The method of claim 1, wherein the effective dose is between 5-50 mg/kg per day.

13. The method of claim 1, wherein the effective dose is between 10-25 mg/kg per day.

14. The method of claim 1, wherein the administration further results in a reduction of lipids in the liver of the individual.

15. The method of claim 1, wherein the administration further results in a reduction in blood triglycerides.

16. The method of claim 1, wherein the administration further results in a reduction in blood LDL level.

17. The method of claim 1, wherein the administration further results in a reduction in blood ALP level.

18. The method of claim 1, wherein the administration further results in a reduction in glycosylated hemoglobin in blood.

19. The method of claim 1, wherein the subject has at least one of steatohepatitis, obesity, type 2 diabetes, and metabolic syndrome.

20. Use of a set of active components in a composition to reduce elevated blood glucose, wherein the composition comprises the set of active components in a therapeutically effective amount, and wherein the set of active components consist essentially of at least two isolated and purified monosaccharides selected from: galacturonic acid, glucuronic acid, galactose, arabinose, rhamnose, xylose, and mannose.

21. The use of claim 20, wherein the set of active components consist essentially of isolated and purified galacturonic acid, isolated and purified galactose and optionally isolated and purified arabinose.

22. The use of claim 20, wherein the molar ratio of galactose to galacturonic acid is between 1 and 3:1, and optionally wherein the molar ratio of arabinose to galacturonic acid is between 4 and 8:1.

23. The use of claim 20, wherein the composition is formulated as a tablet or capsule that includes the set of active components.

24. The use of claim 20, wherein the set of active components comprises at least 70 wt % of the composition.

25. The use of claim 20, wherein the set of active components comprises at least 95 wt % of total active components in the composition.

26. The use of claim 20, wherein each of the isolated and purified monosaccharides has a purity of at least 90% (GC).

27. The use of claim 20, wherein the composition is non-toxic when administered in a rodent model at a dose of 1000 mg/kg/day for a period of four weeks.

28. The use of claim 20, wherein the set of active components consist essentially of at least four isolated and purified monosaccharides selected from: galacturonic acid, glucuronic acid, galactose, arabinose, rhamnose, xylose, and mannose.

29. The use of claim 20, wherein the set of active components consist essentially of at least five isolated and purified monosaccharides selected from: galacturonic acid, glucuronic acid, galactose, arabinose, rhamnose, xylose, and mannose.

30. The use of claim 20, wherein the set of active components consist essentially of isolated and purified galacturonic acid, isolated and purified glucuronic acid, isolated and purified galactose, isolated and purified arabinose, isolated and purified rhamnose, isolated and purified xylose, and isolated and purified mannose.

31. The use of claim 20, wherein the therapeutically effective amount is between 5-50 mg/kg per day.

32. The use of claim 20, wherein the therapeutically effective amount is between 10-25 mg/kg per day.

33. The use of claim 20, wherein administration of the composition further results in a reduction of lipids in the liver of the individual, a reduction in blood triglycerides, a reduction in blood LDL level, a reduction in blood ALP level, and/or a reduction in glycosylated hemoglobin in blood.

34. The use of claim 20, wherein the composition is administered to a subject having at least one of steatohepatitis, obesity, type 2 diabetes, and metabolic syndrome.

35. Use of at least two isolated and purified monosaccharides selected from: galacturonic acid, glucuronic acid, galactose, arabinose, rhamnose, xylose, and mannose in the manufacture of a supplement or therapeutic drug for the reduction of elevated blood glucose.

36. The use of claim 35, wherein the monosaccharides are isolated and purified galacturonic acid, isolated and purified galactose, and isolated and purified arabinose.

37. The use of claim 35, wherein the molar ratio of galactose to galacturonic acid is between 1 and 3:1, and optionally wherein the molar ratio of arabinose to galacturonic acid is between 4 and 8:1.

38. The use of claim 35, wherein the isolated and purified monosaccharides are present in the composition as at least one of a powder and a tablet.

39. The use of claim 35, wherein the therapeutic drug comprises the isolated and purified monosaccharides in an amount of at least 50 wt %.

40. The use of claim 35, wherein the supplement or therapeutic drug is administered in an amount of between 5-50 mg/kg per day.
